(19) United States
(12) Patent Application Publication
(10) Pub. No.: US 2006/0229534 A1
(43) Pub. Date: Oct. 12, 2006

(54) System and method for measuring coefficient variance of resonance frequency of musculoskeletal system

(76) Inventors: Walter Hong-Shong Chang, Jhong-Li City (TW); Chun-Hsiung Shih, Taipei (TW); Hwa-Chang Liu, Taipei (TW); Wen-Jui Huang, Kaohsiung County (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/091,500

(22) Filed: Mar. 29, 2005

(51) Int. Cl. A61B 5/103

(52) U.S. Cl. 600/587

(57) ABSTRACT

The present invention discloses system and method for measuring coefficient variance of resonance frequency of musculoskeletal system. In this regards, the system comprises a signal generation module, a signal retrieval module, and a signal analysis module. The purpose of the system is to measure resonance frequencies of a musculoskeletal system which implanted with an arthroplasty in order to form a statistical sampling space of resonance frequencies. Therefore a coefficient variance of the statistical sampling space of resonance frequencies can be derived. Consequently, the magnitude of this coefficient variance is used for determining whether the implated arthroplasty loose.

### BACKGROUND OF THE INVENTION

1. Field of the Invention

The present invention generally relates to vibration check of medical implant, and more particularly to cohesion check of arthroplasty and musculoskeletal system.

2. Description of the Prior Art

Recently, various kinds of medical implants such as arthroplasty, organs, valves, and cusps are developed. For patients who suffered fracture, arthropathy, or other causes have their arthrosis malfunctioned, arthroplasty implant operation is a common way of treatment, for example, total hip arthorplasty and total knee arthorplasty.

In general, a usual sequela of the arthorplasty implant operation is pains and dyskinesia introduced by the loosening of the implants. One more operation may be required again. Currently, judging the loosening level of arthorplasty relies on X-ray photography, visual observations of patient attitude, and is concluded by subjective judgments of doctor. It heavily depends on personal experience of subjective judgments of doctor; therefore the outcomes are usually just for reference. Besides, some contemporary studies focus on measuring resonance frequency of musculoskeletal systems; take the amplitudes of resonant waves or the number of resonant waves into considerations of clinical judgment of arthorplasty cohesion level. However, personal differences of patients, such as ages, sex, soft tissue thickness, and density of bones, all effects on resonant waves. In consequence, it also requires subjective synthesis of factors mentioned above in measuring resonant waves.

In summarized, there exists a need for a system and method for objectively evaluation of the loosening level of arthorplasty; in order to provide an evidence of further treatment.

### SUMMARY OF THE INVENTION

Therefore, in accordance with the previous summary, objects, features and advantages of the present disclosure will become apparent to one skilled in the art from the subsequent description and the appended claims taken in conjunction with the accompanying drawings.

One objective of the present invention is to disclose a system for measuring coefficient variance of resonance frequency of musculoskeletal system. In this regards, the system comprises a signal generation module, a signal retrieval module, and a signal analysis module. The purpose of the system is to measure resonance frequencies of a musculoskeletal system which implanted with an arthroplasty in order to form a statistical sampling space of resonance frequencies. Therefore a coefficient variance of the statistical sampling space of resonance frequencies can be derived. Moreover, the signal source generation module can further comprise a source generator, an amplifier, and an oscillator to be attached on the arthroplasty-implanted musculoskeletal system. In this regards, the source generator generates oscillatory signals according to instructions from the signal analysis module. Hence, generated oscillatory signals would be amplified and outputted to the oscillator by the amplifier. At last, following the amplified oscillatory signals, the oscillator generates and conducts vibrations into the musculoskeletal system. Besides, the signal sensor module further comprises an accelerometer, to be attached on the arthroplasty-implanted musculoskeletal system, and a charge amplifier. When the musculoskeletal system is vibrating, the accelerometer could sense accelerations of these vibrations. Moreover, the sensed accelerations are amplified by the charge amplifier and feed into the signal analysis module.

Another objective of the present invention is to disclose a method for measuring coefficient variance of resonance frequency of musculoskeletal system. Firstly executing a preparation step, attaching the source generation module and the signal sensor module to proper positions of the musculoskeletal system. In a following vibrating step, the musculoskeletal system is vibrated by the signal source generation module commanded by instructions issued by the signal analysis module. Next, in a signal gathering step, vibrating signals are sensed by the signal sensor module and stored by the signal analysis module. Spectrum transformation techniques are applied on the temporal sensed vibrating signals by the signal analysis module in a resonant frequency calculating step. After calculating the spectral resonant frequency, the outcomes would be store in memorial media. By comparing the quantity of samples with a critical sample space size, if the quantity of samples is not enough, then continue processing the vibrating step; otherwise, a coefficient variance analysis step would be executed. After calculating the coefficient variance, a decision step would be processing by comparing this coefficient variance with a cohesion threshold. If the coefficient variance is larger than the cohesion threshold, it implies that the musculoskeletal system of measured patient is loosen; otherwise, cohesive well.

Another objective of the present invention is to evaluate the differential level of musculoskeletal cohesion between a loosened group and a non-loosened group. Therefore, the disclosed system and method can be adapted to exploit this evaluation process. According to structural theories, if a whole system is bounded cohesive, a stable resonant frequency of the whole system could be measured; otherwise, the resonant frequency of the whole system would be drifted. Following the deduction above, a system and method is disclosed by the present invention to objectively and concisely evaluate the loosening level of anthroplasty cohesion, in order to provide a concrete evidence for further treatment.

### BREIF DESCRIPTION OF THE DRAWINGS

The accompanying drawings incorporated in and forming a part of the specification illustrate several aspects of the present invention, and together with the description serve to explain the principles of the disclosure. In the drawings:

FIG. 1 is a block diagram illustrates a system for measuring coefficient variance of resonance frequency of musculoskeletal system in accordance with an embodiment of the present invention; and

FIG. 2 is a flowchart diagram of the system shown in FIG. 1.

### DETAILED DESCRIPTION OF THE PREFERRED EMBODIMENTS

The present disclosure can be described by the embodiments given below. It is understood, however, that the embodiments below are not necessarily limitations to the present disclosure, but are used to a typical implementation of the invention.

Having summarized various aspects of the present invention, reference will now be made in detail to the description of the invention as illustrated in the drawings. While the invention will be described in connection with these drawings, there is no intent to limit it to the embodiment or embodiments disclosed therein. On the contrary the intent is to cover all alternatives, modifications and equivalents included within the spirit and scope of the invention as defined by the appended claims.

It is noted that the drawings presents herein have been provided to illustrate certain features and aspects of embodiments of the invention. It will be appreciated from the description provided herein that a variety of alternative embodiments and implementations may be realized, consistent with the scope and spirit of the present invention.

It is also noted that the drawings presents herein are not consistent with the same scale. Some scales of some components are not proportional to the scales of other components in order to provide comprehensive descriptions and emphasizes to this present invention.

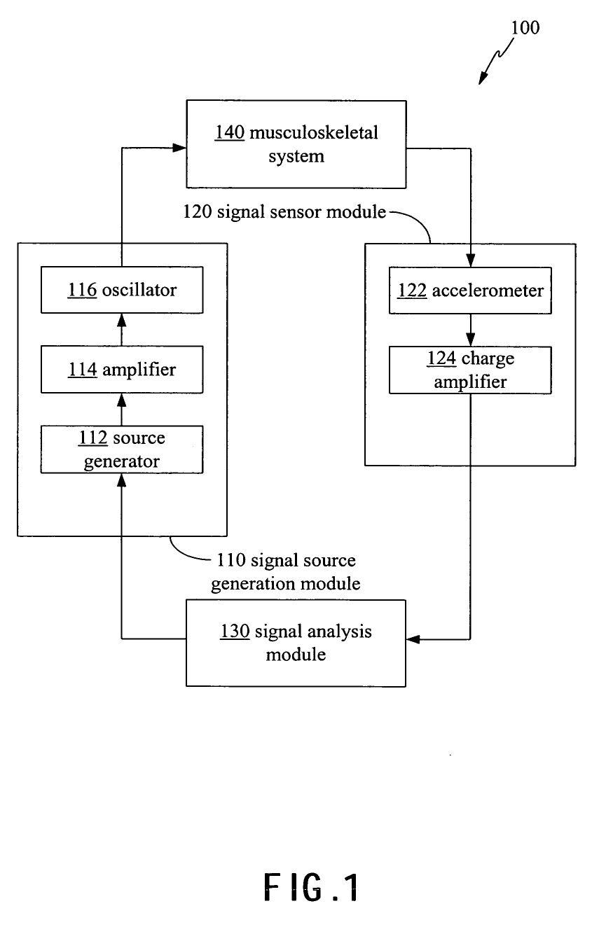

Please refer to FIG. 1, which illustrates a system 100 for measuring coefficient variance of resonance frequency of musculoskeletal system in accordance with an embodiment of the present invention. In this regards, the system 100 comprises a signal source generation module 110, a signal sensor module 120, and a signal analysis module 130. The system 100 is used for measuring resonant signals of an arthroplasty-implanted musculoskeletal system 140, forming a statistical sampling space of measured resonant signals, and calculating a coefficient variance of the sampling space in the end.

In a better example of the embodiment, the arthroplasty-implanted musculoskeletal system 140 is a compound system implanted a total hip arthroplasty. Furthermore, the compound system comprises a femur, a great trochanter, and a acettabulum. In another example of the present embodiment, the arthroplasty-implanted musculoskeletal system 140 is a compound system implanted a knee arthroplasty. In other words, the present invention could apply to various musculoskeletal systems.

As shown in FIG. 1, the signal source generation module 110 further comprises a source generator 112, an amplifier 114, and an oscillator 116 to be attached on the arthroplasty-implanted musculoskeletal system 140. In this regards, the source generator 112 generates oscillatory signals according to instructions from the signal analysis module 130. Hence, generated oscillatory signals would be amplified and outputted to the oscillator 116 by the amplifier 114. At last, following the amplified oscillatory signals, the oscillator 116 generates and conducts vibrations into the musculoskeletal system 140.

As shown in FIG. 1, the signal sensor module 120 further comprises an accelerometer 122, to be attached on the arthroplasty-implanted musculoskeletal system 140, and a charge amplifier 124. When the musculoskeletal system 140 is vibrating, the accelerometer 122 could sense accelerations of these vibrations. Moreover, the sensed accelerations are amplified by the charge amplifier 124 and feed into the signal analysis module 130.

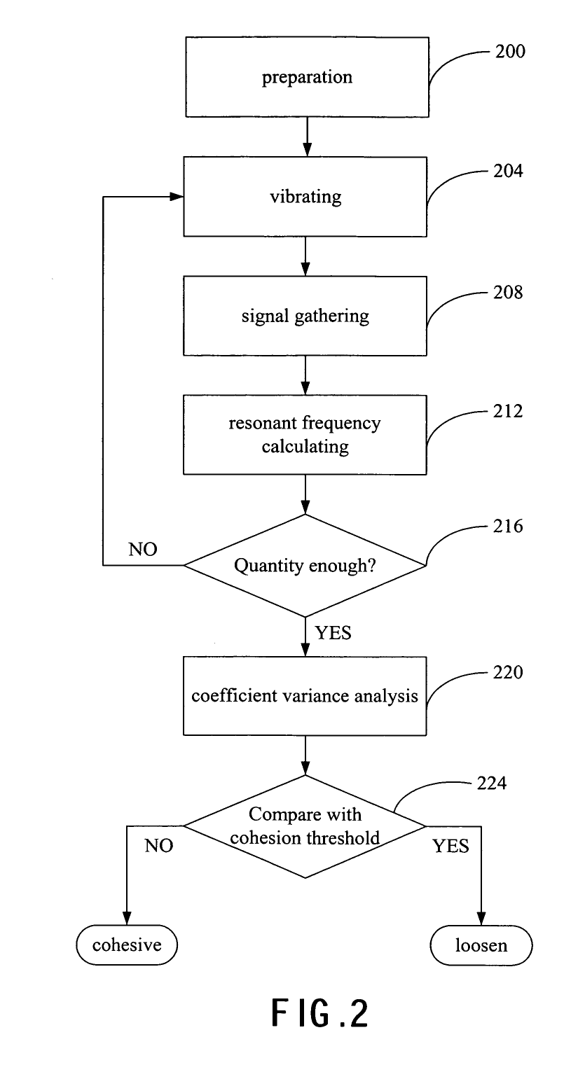

Please refer to FIG. 2, which shows a flowchart diagram of the system 100 shown in FIG. 1. Firstly executing a preparation step 200, attaching the source generation module 110 and the signal sensor module 120 of the system 100 to proper positions of the musculoskeletal system 140. In a better example of the present embodiment, the arthroplasty-implanted musculoskeletal system 140 is a total hip arthroplasty-implanted musculoskeletal compound system. In this regards, the oscillator 116 of the source generation module 110 would be placed at the lateral femoral condyle. Besides, the accelerometer 122 would be put at the great trochanter. In this embodiment, the patient who is measured by this system 100 can lie flatly, lie sidely, stand, or post any other attitudes.

Processing a vibrating step 204 after the preparation step 200, instructions issued by the signal analysis module 130 are sent to the signal source generation module 110. Hence the musculoskeletal system 140 is vibrated by the signal source generation module 110. Moreover, in a signal gathering step 208, vibrating signals are sensed by the signal sensor module 120 and stored by the signal analysis module 130. Next, processing a resonant frequency calculating step 212, spectrum transformation techniques are applied on the temporal sensed vibrating signals by the signal analysis module 130. After calculating the spectral resonant frequency, the outcomes would be store in memorial media. In this regards, the spectrum transformation techniques are referred to well-known linear or non-leaner transformation techniques, such as fast Fourier transformation or wavelet transformation.

Next, in a decision step 216, determining whether enough resonant frequency samples are accumulated by comparing the quantity of samples with a critical sample space size. If the quantity of samples is not enough, then continue processing the vibrating step 204; otherwise, a coefficient variance analysis step 220 would be executed. In a better example of the present embodiment, the critical sample space size is a predetermined value. Besides, the sample space is measured at a single attitude of the patient.

In this coefficient variance analysis step 220, a first formula is calculated as a mean (\overline{X}). Next, a second formula is put into consideration of a standard deviation (SD). A statistical coefficient variance (CV) is approached by applying a third formula at the last. In these three formulas, N is denoted as the quantity of resonant frequency samples, and Xᵢ is represented as the i-th sample. The mean shown in the first formula is a quotient of a sum of resonant frequency samples divided by the quantity of resonant frequency samples. The standard deviation shown in the second formula is a square root of a quotient minus one (1); moreover, the quotient is calculated as a differential, a sum of squares of samples minus a product of the mean and the quantity, divided by the quantity. At last, the coefficient variance shown in the third formula is a quotient as the standard deviation divided by the means.

$$\overline{X} = \frac{{\sum }_{i = 1}^{N} {X}_{i}}{N} first formula$$

$$SD = \sqrt{\frac{{\sum }_{i = 1}^{N} {X}_{i}^{2} - N {\overline{X}}^{2}}{N} - 1} second formula$$

$$CV = \frac{SD}{\overline{X}} third formula$$

After calculating the coefficient variance, a decision step 224 would be processing by comparing this coefficient variance with a cohesion threshold. If the coefficient variance is larger than the cohesion threshold, it implies that the musculoskeletal system 140 of measured patient is loosen; otherwise, cohesive well. In this regards, the cohesion threshold is a given value according to the measured part of musculoskeletal system 140 and the measured attitude of patient.

In statistics, coefficient variance is used to evaluate the differential level of a common character between different groups. An objective of the present invention is to evaluate the differential level of musculoskeletal cohesion between a loosened group and a non-loosened group. Therefore, the disclosed system and method can be adapted to exploit this evaluation process. According to structural theories, if a whole system is bounded cohesive, a stable resonant frequency of the whole system could be measured; otherwise, the resonant frequency of the whole system would be drifted. Following the deduction above, the inventors applied the disclosed system and method to measure patients who implanted total hip anthroplasty. The coefficient variances measured on a cohesive well group are distributed between 0.022 and 0.035, and the values measured on a loosened group are distributed around 0.035 and 0.061. Taking 0.035 as a proper cohesion threshold, whether the measured musculoskeletal system is loosen or not can be determined concisely.

The foregoing description is not intended to be exhaustive or to limit the invention to the precise forms disclosed. Obvious modifications or variations are possible in light of the above teachings. In this regard, the embodiment or embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the inventions as determined by the appended claims when interpreted in accordance with the breath to which they are fairly and legally entitled.

It is understood that several modifications, changes, and substitutions are intended in the foregoing disclosure and in some instances some features of the invention will be employed without a corresponding use of other features. Accordingly, it is appropriate that the appended claims be construed broadly and in a manner consistent with the scope of the invention.

1. A measurement system for measuring a coefficient variance of resonant frequency of a musculoskeletal system, comprising:
a signal source generation module accepts instructions from a signal analysis module to generate vibration signals to said musculoskeletal system;
a signal sensor module senses vibration signals from said musculoskeletal system and feed into said signal analysis module; and
said signal analysis module calculates said coefficient variance of resonant frequency of said musculoskeletal system according to vibration signals fed by said signal sensor module.

2. A measurement system of claim 1, wherein said signal source generation module further comprises a source generator, an amplifier, and an oscillator attached on said musculoskeletal system, said source generator generates oscillatory signals according to instructions from said signal analysis module, generated oscillatory signals are amplified and outputted to said oscillator by said amplifier, said oscillator generates and conducts vibrations into said musculoskeletal system following the amplified oscillatory signals.

3. A measurement system of claim 1, wherein said signal sensor module further comprises an accelerometer attached on said musculoskeletal system and a charge amplifier, accelerations of said musculoskeletal system vibrations are sensed by said accelerometer, amplified by said charge amplifier, and be fed into said signal analysis module.

4. A measurement system of claim 1, wherein said signal analysis module applies a linear or non-linear spectrum transformation technique to transform temporal vibration signals from said signal sensor module into spectral resonant frequencies and store the spectral resonant frequencies in memorial media.

5. A measurement system of claim 4, wherein said linear spectrum transformation technique is fast Fourier transformation.

6. A measurement system of claim 4, wherein said non-linear spectrum transformation technique is wavelet transformation.

7. A measurement system of claim 4, said signal analysis module further comprises a comparison a quantity of resonant frequency samples in memorial media with a critical sample space size, continuing collecting resonant frequency samples whenever said quantity is insufficient.

8. A measurement system of claim 7, wherein said signal analysis module further comprises calculating said coefficient variance whenever said comparison indicated that said quantity is sufficient.

9. A measurement system of claim 8, wherein said signal analysis module further comprises calculating a standard deviation and a mean, said coefficient variance is a quotient of said standard deviation and said mean.

10. A measurement system of claim 9, wherein said mean is a quotient of a sum of resonant frequency samples divided by said quantity of resonant frequency samples.

11. A measurement system of claim 9, wherein said standard deviation is a square root of a quotient minus one (1), the quotient is calculated as a differential, a sum of squares of samples minus a product of the mean and said quantity, divided by said quantity.

12. A measurement system of claim 8, wherein said signal analysis module further comprises comparing said coefficient variance with a cohesion threshold, said musculoskeletal system is determined as loosen whenever said coefficient variance is larger than said cohesion threshold, said musculoskeletal system is contrarily determined as cohesive well whenever said coefficient variance is not larger than said cohesion threshold.

13. A measurement system of claim 13, wherein said musculoskeletal system is a total hip anthroplasty implanted musculoskeletal system.

14. A measurement system of claim 14, wherein said cohesion threshold is 0.035.

15. A measurement method for measuring a coefficient variance of resonant frequency of a musculoskeletal system, comprising:
providing a measurement system, which further comprises a signal source generation module attached to said musculoskeletal system, a signal sensor module attached to said musculoskeletal system, and a signal analysis module;
performing a vibrating step, wherein said vibrating step let said signal source generation module accepts instructions from said signal analysis module to generate vibration signals to said musculoskeletal system;
performing a gathering step, wherein said gathering step let said signal sensor module senses vibration signals from said musculoskeletal system and feed into said signal analysis module; and
performing a analysis step, wherein said analysis step let said signal analysis module calculates said coefficient variance of resonant frequency of said musculoskeletal system according to vibration signals fed by said signal sensor module.

16. A measurement method of claim 15, wherein said signal source generation module further comprises a source generator, an amplifier, and an oscillator attached on said musculoskeletal system, said source generator generates oscillatory signals according to instructions from said signal analysis module, generated oscillatory signals are amplified and outputted to said oscillator by said amplifier, said oscillator generates and conducts vibrations into said musculoskeletal system following the amplified oscillatory signals.

17. A measurement method of claim 15, wherein said signal sensor module further comprises an accelerometer attached on said musculoskeletal system and a charge amplifier, accelerations of said musculoskeletal system vibrations are sensed by said accelerometer, amplified by said charge amplifier, and be fed into said signal analysis module.

18. A measurement method of claim 15, wherein said analysis step further comprises that said signal analysis module applies a linear or non-linear spectrum transformation technique to transform temporal vibration signals from said signal sensor module into spectral resonant frequencies and store the spectral resonant frequencies in memorial media.

19. A measurement method of claim 18, wherein said linear spectrum transformation technique is fast Fourier transformation.

20. A measurement method of claim 18, wherein said non-linear spectrum transformation technique is wavelet transformation.

21. A measurement method of claim 15, wherein said analysis step further comprises that said signal analysis module compares a quantity of resonant frequency samples in memorial media with a critical sample space size, continuing collecting resonant frequency samples whenever said quantity is insufficient.

22. A measurement method of claim 21, wherein said analysis step further comprises that said signal analysis module calculates said coefficient variance whenever said comparison indicated that said quantity is sufficient.

23. A measurement method of claim 22, wherein said analysis step further comprises that said signal analysis module calculates a standard deviation and a mean, said coefficient variance is a quotient of said standard deviation and said mean.

24. A measurement method of claim 23, wherein said mean is a quotient of a sum of resonant frequency samples divided by said quantity of resonant frequency samples.

25. A measurement method of claim 23, wherein said standard deviation is a square root of a quotient minus one (1), the quotient is calculated as a differential, a sum of squares of samples minus a product of the mean and said quantity, divided by said quantity.

26. A measurement method of claim 22, wherein said analysis step further comprises that said signal analysis module compares said coefficient variance with a cohesion threshold, said musculoskeletal system is determined as loosen whenever said coefficient variance is larger than said cohesion threshold, said musculoskeletal system is contrarily determined as cohesive well whenever said coefficient variance is not larger than said cohesion threshold.

27. A measurement method of claim 26, wherein said musculoskeletal system is a total hip anthroplasty implanted musculoskeletal system.

28. A measurement method of claim 27, wherein said cohesion threshold is 0.035.
